(19) United States
(12) Patent Application Publication
(10) Pub. No.: US 2024/0091055 A1
(43) Pub. Date: Mar. 21, 2024

(54) Heated garment for period pain

(71) Applicant: Sorascape, Inc., Rancho Palos Verdes, CA (US)

(72) Inventors: Julia Layton, Rancho Palos Verdes, CA (US); Jenny Pan, San Gabriel, CA (US)

(73) Assignee: Sorascape, Inc., Rancho Palos Verdes, CA (US)

(21) Appl. No.: 18/468,451

(22) Filed: Sep. 15, 2023

Related U.S. Application Data
(60) Provisional application No. 63/407,147, filed on Sep. 15, 2022.

(51) Int. Cl. A61F 7/02

(52) U.S. Cl. CPC A61F 7/02; A61F 2007/0071

(57) ABSTRACT

Apparatus is provided for garments to relieve period pain. The apparatus presented comprises a garment, one or more sets of heating elements or fabric with heating fibers, a power source, and a controller. A user may use the apparatus to relieve period pain. The apparatus permits a user to provide long-lasting and durable relief of period pain, while attending to typical daily routines, without the need for medication, bulky heating devices, or inconvenience. The present invention solves problems with the currently available means and apparatuses of relieving pain from periods.

### FIELD OF THE INVENTION

The presently disclosed subject matter relates to providing apparatus for relief of menstrual period pain, and more particularly, to apparatus for garments to relieve period pain.

### BACKGROUND OF THE INVENTION

Period pain, that is, pain associated with menstruation and menstrual cramps, exists for half of the population, for a significant percentage of people's lives. Menstrual pain, clinically termed dysmenorrhea, has symptoms which commonly include pain, throbbing, cramping, aches, and more. Other devices and treatments exist for period pain, which all have various shortcomings. People have used heating pads to relieve the pain and cramping. Shortcomings of this approach include the need to be near an electric outlet, and thus the inability to move around and go through one's normal daily routine and work. In addition, holding a heating pad in place is not necessarily simple, and multiple heating pads may be needed to reach areas of the person and provide relief. The use of hot water bottles has similar limitations: the need for hot water, the inability to go about one's daily routine, the difficulty in applying the heat to the right places, meaning areas of the person's body, to provide relief, and the need to replenish the hot water. In addition, a hot water bottle cannot provide a consistent amount of heat: they are hottest when filled, and cool off during the time one uses them. The same is true of heating pads that can be warmed mechanically, such as in a hot water bath or in a microwave oven. Chemical heat packs, similarly, can offer heat and relief, but the amount of heat they provide is not controllable, and tapers over the duration in which they provide heat and one uses them. In addition, they may be hard to place and relocate while using them.

Medicated solutions exist, such as pain killers, but require the person to take the drugs, which not everyone wants to do or can tolerate. Massage and acupuncture can offer relief, but they are relatively costly, inconvenient—as one needs to schedule a visit, and typically visit the provider—and require an interruption in one's daily routine.

Finally, the prior art does not allow a user to easily reuse and refill or recharge a heating solution, while wearing or using something that is hidden from view of others, and allows the user to go about their daily routine.

### SUMMARY OF THE INVENTION

The present disclosure meets all these needs, by disclosing apparatus that provides heating elements for discrete and low-profile heating, where the heating elements are user-controllable and in a washable, reusable, and rechargeable garment.

The present disclosure addresses the problems of the prior art, which do not present apparatus for washable, reusable, and rechargeable garments that can provide directed heating for period paid while allowing the user to go about her daily routine. Unlike heating pads, the user does not have to stay near an electrical outlet, and can go about her daily routine. Unlike hot water bottles, heated packs, or chemical heating packs, the amount of heat is steady, can last longer than the prior art, and is discrete enough that it can be worn and carried on the user's person, while the user goes about her daily routine and life. Unlike medications, the user does not need to take any drugs.

Further, the present disclosure introduces a product that allows the user to control the heat and the relief she experiences, while not appearing to be carrying, and without a need to carry, any particular devices, in a form that can be worn under any clothes. The apparatus of the present disclosure can be washed and reused, and can be recharged. In some aspects of the present disclosure, the apparatus can be charged and powered by any of a range of commercially available batteries.

In another aspect, the apparatus comprises a garment to relieve period pain. The apparatus can be made as any of a range of types of garments.

These aspects of the present invention, and others disclosed in the Detailed Description of the Drawings, represent improvements on the current art. This summary is provided to introduce a selection of concepts in a simplified form that are further described below in the Detailed Description of the Drawings. This Summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of various aspects, is better understood when read in conjunction with the appended drawings. For the purposes of illustration, the drawings show exemplary aspects; but the presently disclosed subject matter is not limited to the specific methods and instrumentalities disclosed. In the drawings, like reference characters generally refer to the same components or steps of the device throughout the different figures. In the following detailed description, various aspects of the present invention are described with reference to the following drawings, in which:

FIG. 1 shows a front elevation view of an aspect of the apparatus of the present disclosure.

FIG. 2 shows a rear elevation view of an aspect of the apparatus of the present disclosure.

FIG. 3 shows a first side elevation view of an aspect of the apparatus of the present disclosure.

FIG. 4 shows a second side elevation view of an aspect of the apparatus of the present disclosure.

FIG. 5 shows an elevation view of an aspect of the apparatus of the present disclosure.

FIG. 6 shows a front and top perspective view of an aspect of the apparatus of the present disclosure.

FIG. 7 shows a rear elevation view of an aspect of the apparatus of the present disclosure.

FIG. 8 shows a front elevation view of an aspect of the apparatus of the present disclosure.

FIG. 9 shows a rear elevation view of an aspect of the apparatus of the present disclosure.

FIG. 10A shows a front elevation view of an aspect of the apparatus of the present disclosure in an underwear garment.

FIG. 10B shows a rear elevation view of an aspect of the apparatus of the present disclosure in the underwear garment of FIG. 10A.

FIG. 10C shows a front elevation view of an aspect of the apparatus of the present disclosure in a leggings garment.

FIG. 10D shows a rear elevation view of an aspect of the apparatus of the present disclosure in the leggings garment of FIG. 10C.

FIG. 10E shows a front elevation view of an aspect of the apparatus of the present disclosure in a shirt garment.

FIG. 10F shows a front elevation view of an aspect of the apparatus of the present disclosure in a jacket garment.

FIG. 10G shows a rear elevation view of an aspect of the apparatus of the present disclosure in the jacket garment of FIG. 10F.

FIG. 11 shows a schematic view of an aspect of the apparatus of the present invention.

### DETAILED DESCRIPTION OF THE DRAWINGS

The presently disclosed invention is described with specificity to meet statutory requirements. But, the description itself is not intended to limit the scope of this patent. Rather, the claimed invention might also be configured in other ways, to include different steps or elements similar to the ones described in this document, in conjunction with other present or future technologies. Moreover, although the term “step” or similar terms may be used herein to connote different aspects of methods employed, the term should not be interpreted as implying any particular order among or between various steps herein disclosed unless and except when the order of individual steps is explicitly described. The word “approximately” as used herein means within 5% of a stated value, and for ranges as given, applies to both the start and end of the range of values given.

In the following description, numerous specific details are set forth to provide a thorough understanding of the invention. But, the present invention may be practiced without these specific details. Structures and techniques that would be known to one of ordinary skill in the art have not been shown in detail, in order not to obscure the invention. Referring to the figures, it is possible to see the various major elements constituting the apparatus and methods of use the present invention.

The present invention comprises an apparatus 100 for heating relief of period pain. At a high level of overview, the present disclosure provides garments to relieve period pain. The garments of the present disclosure may be advantageously made for and/or used for heating or warming purposes not specific to period pain, including but not limited to heating a person, relief of any pain, relief of cold weather or cold locations or cold rooms or workplaces, or injury prevention such as by warming up muscles and parts of the body.

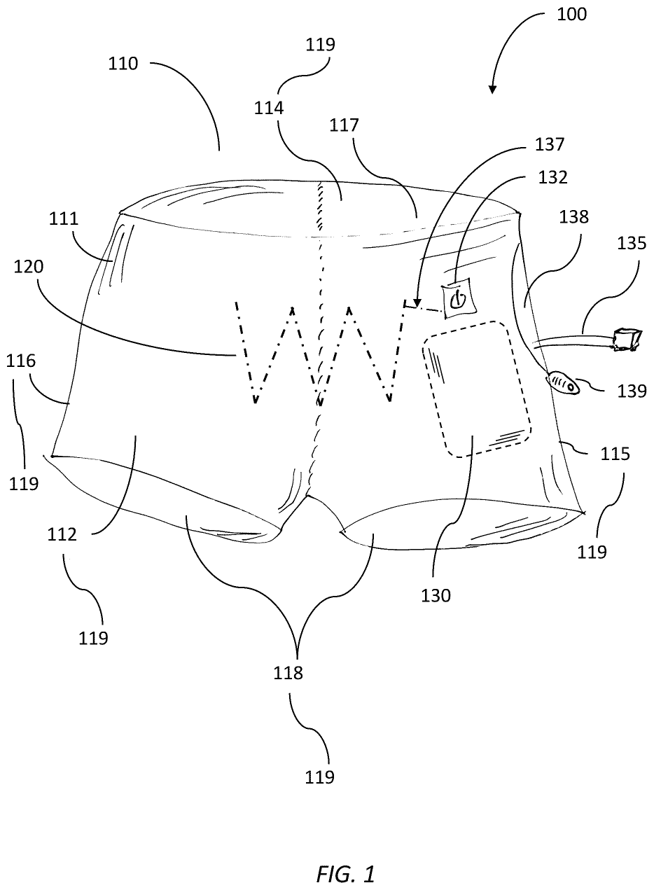

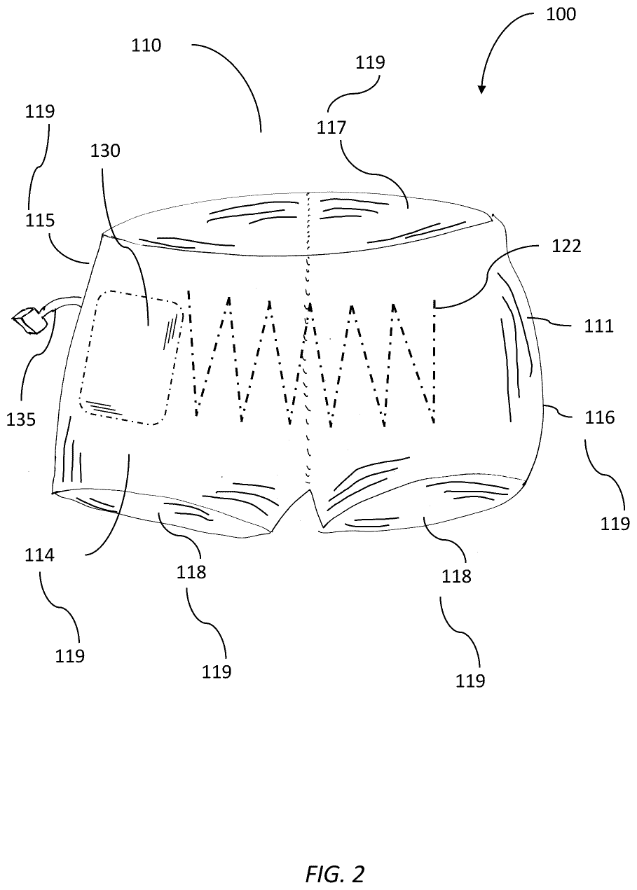

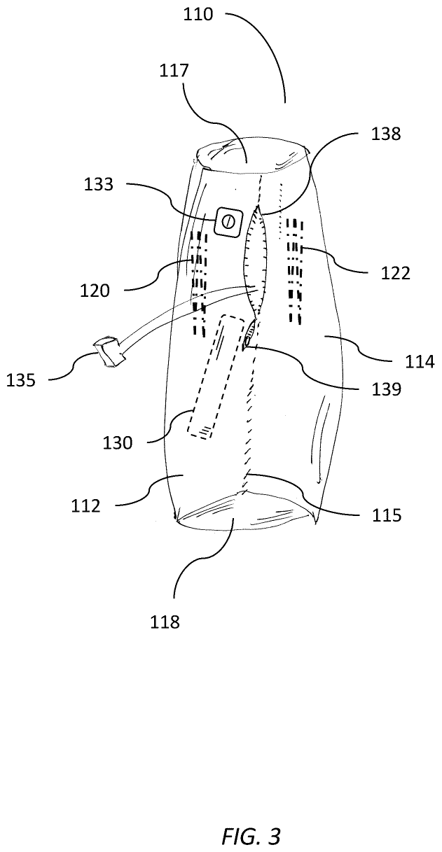

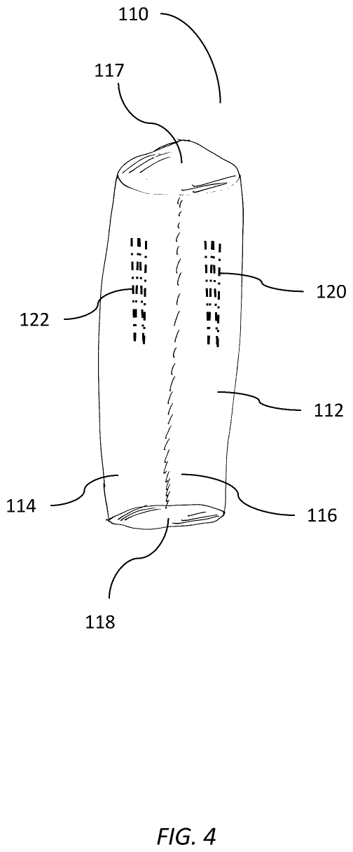

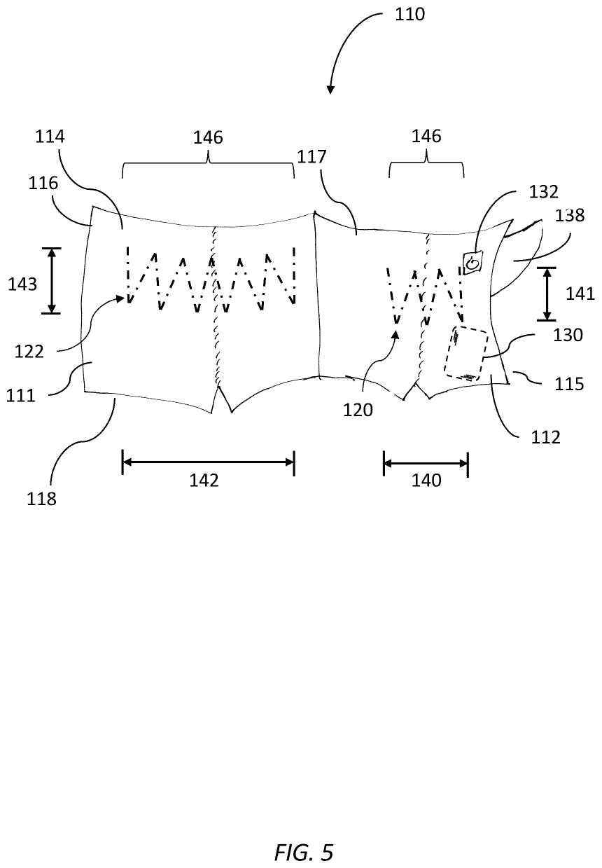

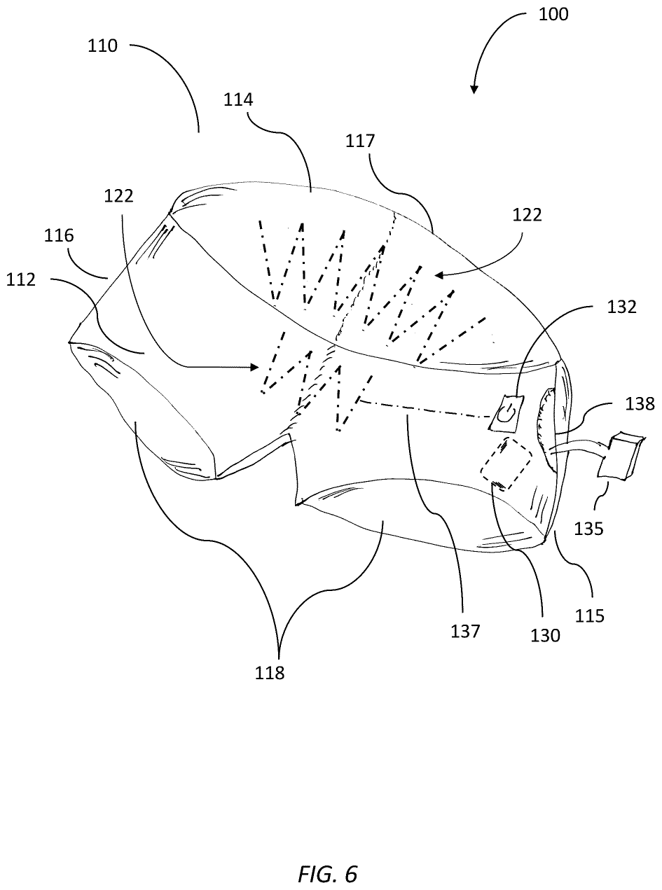

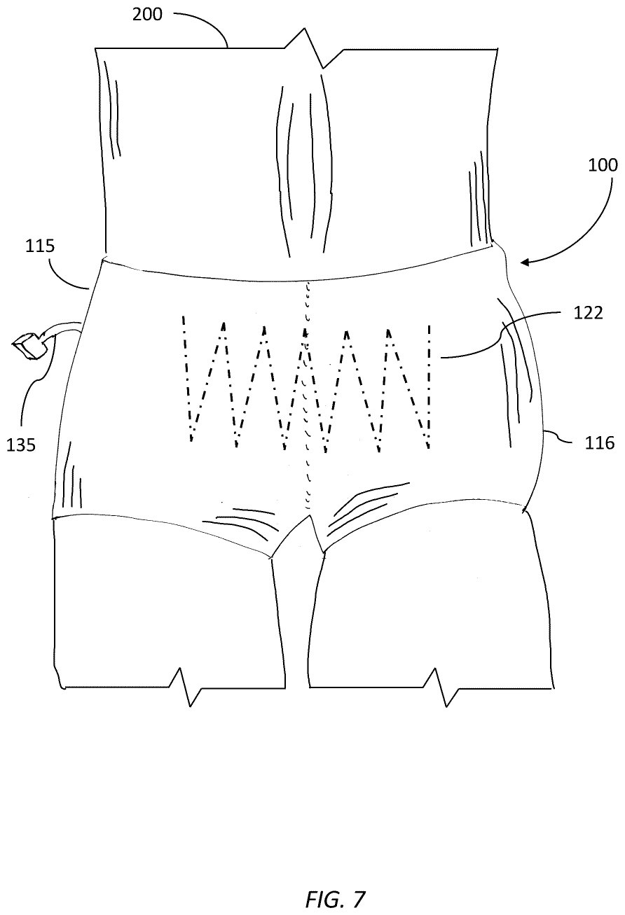

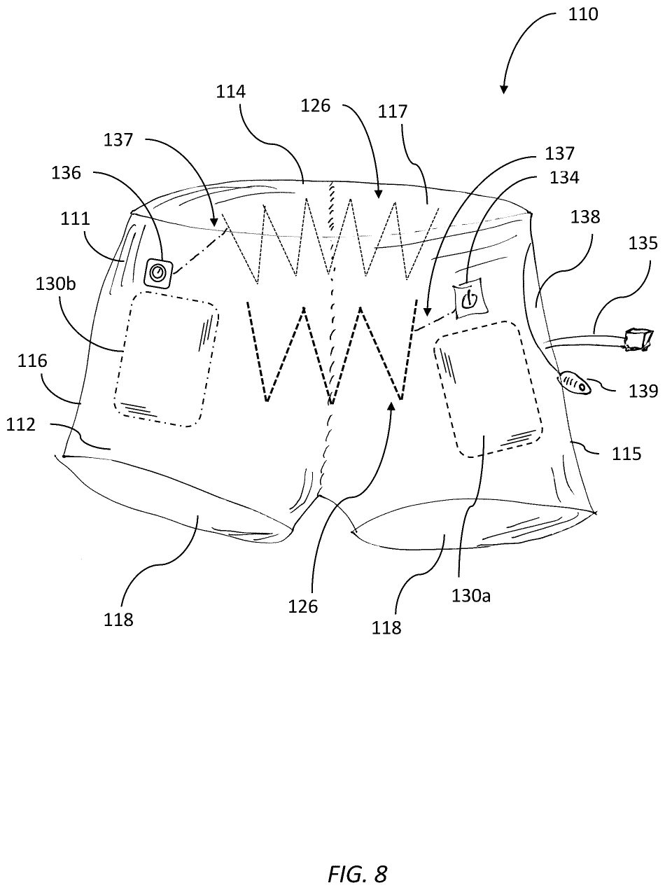

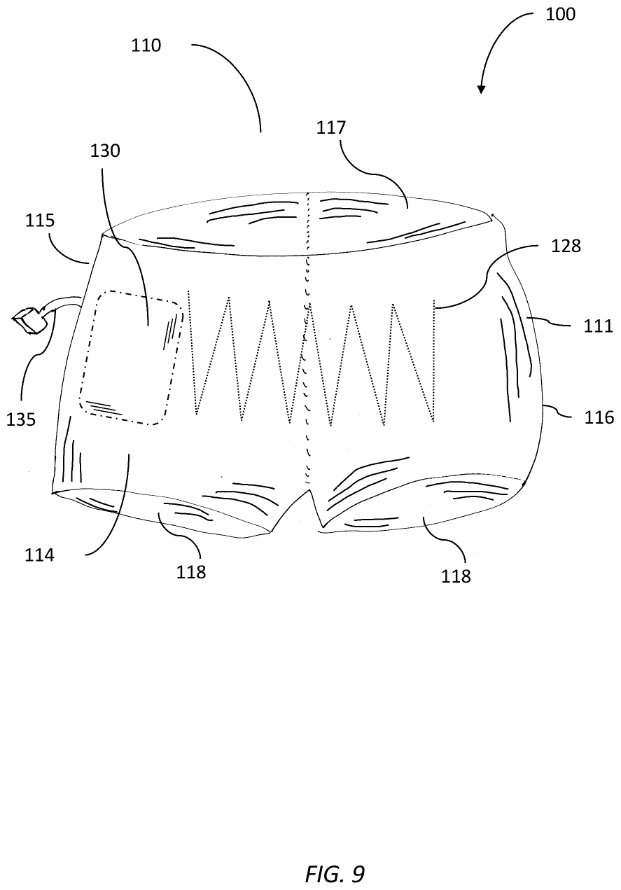

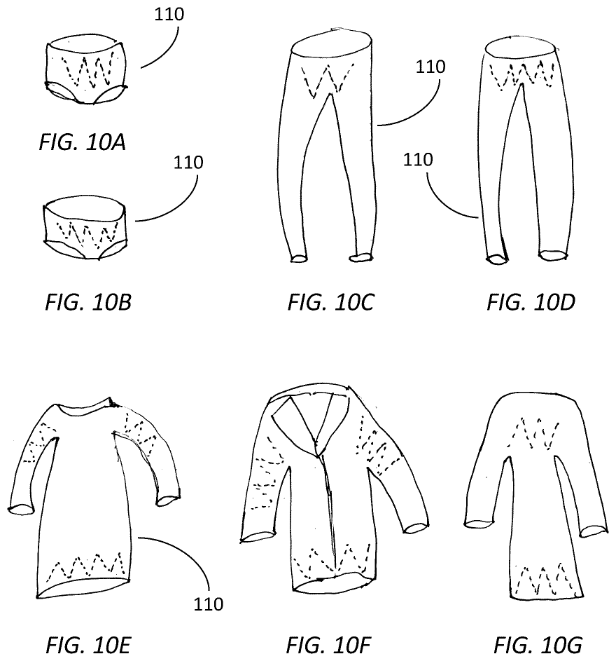

With reference to FIG. 1, FIG. 2, FIG. 3, FIG. 4, FIG. 6, FIG. 7, FIG. 8, FIG. 9, and FIG. 10A-10G, the apparatus 100 of the present invention comprises a garment 110. The garment 110 may comprise shorts, as illustrated in FIGS. 1-9, underwear, as illustrated in FIG. 10A and FIG. 10B, camisoles, leotards, or a bodysuit or unitard, pants, leggings, as illustrated in FIG. 10C and FIG. 10D, bathing suits, wetsuits, shirts, as illustrated in FIG. 10E, sweatshirts, jackets, as illustrated in FIG. 10F and FIG. 10G, or other types of apparel, whether listed and illustrated herein or not. The garment 110 comprises a fabric 111, as the garment is made of at least one fabric. In some aspects of the present disclosure, all of the garment 110 is made from the fabric 111, being just one type of fabric. In other aspects of the present disclosure, at least a portion of the garment 110 is made with the fabric 111, and other portions, parts, or areas of the garment 110 are made from different types of fabric or other materials. The garment 110 comprises at least a front side 112, a back side 114, a left 115, a right 116, a top side 117, and a bottom side 118, collectively a set of one or more sides 119. In some aspects of the present disclosure, one or more of the forgoing sides of the garment 110 may be minimal or nonexistent, as in the garment 110 depicted in FIG. 1 as a pair of shorts, which may effectively not have a top side 117 other than an edge. In other aspects, such as a camisole, shirt, sweatshirt, or jacket, there may effectively not be a bottom side 118 other than an edge.

With reference to FIGS. 1-9 and FIGS. 10A-10G, the garment 110 comprises a first set of heating channels 120. The garment 110 may comprise a second set of heating channels 122. In some aspects of the present disclosure, the garment 110 may comprise any number of sets of heating channels, typically two or more sets of heating channels. The first set of heating channels 120 may be located in the front side 112 of the garment 110. In aspects of the present disclosure with the second set of heating channels 122, the second set of heating channels 122 may be located in the back side 114. Any of the foregoing sets of heating channels, which may be referred to as heating elements, regardless of the number of such sets of heating channels, may be disposed or located in any of the set of one or more sides 119. Advantageously, any of the foregoing sets of heating channels may be disposed to be positioned near to the lower abdomen region of a user 200, to relieve pain in or near the uterus and/or uterine muscles. In some aspects of the present disclosure, and with reference to FIGS. 1-9 and FIGS. 10A-10G, some of the foregoing sets of heating channels may be positioned over some or all of the person's lumbar region, abdomen, chest, back, sides, hips, and/or thighs.

With further reference to FIG. 8 and FIG. 9, any of the first set of heating channels 120, the second set of heating channels 122, and if present, the other sets of heating elements may comprise carbon fiber materials 126, a material that advantageously provides flexibility in the garment 110 and the ability to wash the garment 110, while functioning efficiently as heating elements for more effective heating and relief of menstrual pain. Furthermore, the use of carbon fiber materials 126 for the first set of heating channels 120, the second set of heating channels 122, and any other sets of heating elements allow the use of a range of voltage levels applied to the carbon fiber materials 126 to generate an advantageous level of heat for pain relief, as desired by the user 200 at an advantageously low level of power consumption. In some aspects of the present disclosure, any of the first set of heating channels 120, the second set of heating channels 122, or any other sets of heating elements may comprise metallic thread 128 and/or carbon fiber materials 126 and/or other materials now known or later invented. Any or all of the first set of heating channels 120, the second set of heating channels 122, and if present, the other sets of heating elements may be arranged in a pattern 146, wherein the pattern 146 may be a zig-zag pattern, as shown in FIGS. 1-9, or a sinusoidal pattern or in another pattern or patterns, among and/or in the material of the garment 110. The pattern 146 serves to provide an effective area of heating for the user 200, so that heat is delivered to the user 200 where it can provide effective relief of menstrual pain. The pattern 146 of the first set of heating channels 120, meaning the arrangement of the first set of heating channels 120 within the garment 110, may be implemented to provide even heating across an area of the body of the user 200, or may be implemented to provide a greater amount of heating in some areas of the body of the user 200 and a lesser amount of heating in other areas of the of the body of the user 200. Additionally, the pattern 146 of the first set of heating channels 120 may, advantageously, provide stretchiness to the garment 110 such that the garment 110 can flex and move with the movements of the user 200, and such that the garment 110 can be washed in typical home or commercial clothes-washing machines, providing an advantage in convenience and hygiene over the prior art.

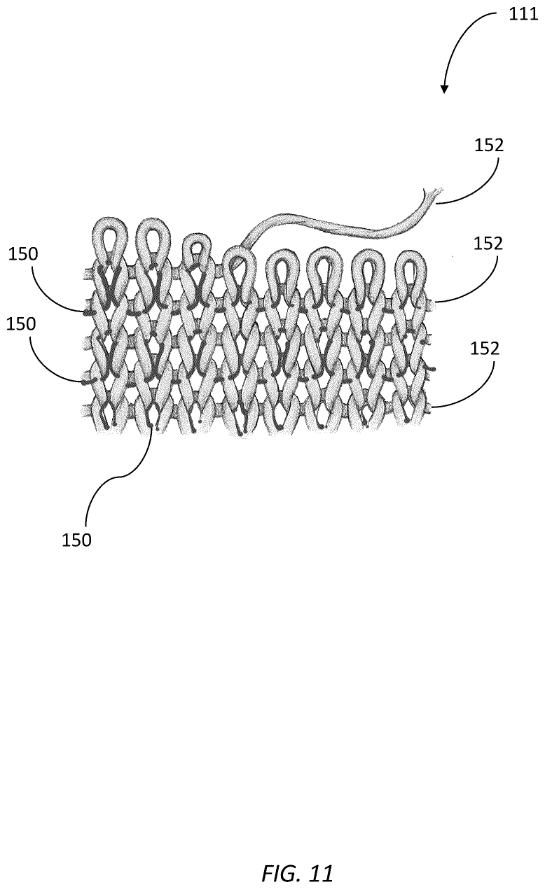

With further reference to FIG. 11, in some aspects of the present disclosure, the garment 110 may have heating fibers 150 woven or otherwise made or attached into some or all of the fabric 111, comprising non-heating fibers 152, or any materials used in making the garment 110. The heating fibers 150 may be in addition to the first set of heating channels 120 or any number of sets of heating channels, or the heating fibers 150 may be used alone, without any heating channels. The heating fibers 150 may comprise materials selected from a group including but not limited to carbon fiber materials 126, a metallic thread 128 including but not limited to silver thread, or other conductive and resistive materials now known or later invented which are suitable for inclusion in the fabric 111, are flexible and washable, and can be powered to emit heat. In such an aspect of the present disclosure, one or more areas of the garment 110 could emit heat, with different areas of the garment 110 woven with different amounts of the heating fibers 150 (per unit area) and/or different or multiple types of heating fibers 150, such that the different areas of the garment 110 emit different amounts of heat to the body of the user 200. Such aspects of the present disclosure, with heating fibers 150 integrated into the fabric 111, or into more than one fabric 111 in aspects of the garment 110 with more than one fabric 111, provide other advantages of aspects of the present disclosure with heating channels, including but not limited to flexibility of the garment 110, washability of the garment 110, and discreetness of the garment 110 as a solution to menstrual pain that is portable, effective, simple to use over a desired and long extent of time, and not noticeable to others.

With reference to FIGS. 1-9 and FIGS. 10A-10G, any of the first set of heating channels 120, the second set of heating channels 122, or any other sets of heating elements, and/or any of the areas of the fabric 111 of the garment 110 wherein the fabric 111 comprises heating fibers 150, may be arranged, extended, or placed in one or more regions of the garment 110. The first set of heating channels 120, or heating fibers 150, when placed in or on the front side 112, may have a front horizontal extent 140 of approximately 2 inches to 10 inches, or the front horizontal extent 140 may comprise approximately 10% to 90% of a total horizontal extent of the front side 112, or may comprise approximately the entire total horizontal extent of the front side 112. The first set of heating channels 120, or heating fibers 150, when placed in or on the front side 112, may have a front vertical extent 141 of approximately 2 inches to 6 inches, or the front vertical extent 141 may comprise approximately 10% to 90% of a total vertical extent of the front side 112, or may comprise approximately the entire total vertical extent of the front side 112. The second set of heating channels 122, or heating fibers 150, when placed in or on the back side 114, may have a back horizontal extent 142 of approximately 2 inches to 14 inches, or the back horizontal extent 142 may comprise approximately 10% to 90% of a total horizontal extent of the back side 114, or may comprise approximately the entire total horizontal extent of the back side 114. The second set of heating channels 122, or heating fibers 150, when placed in or on the back side 114, may have a back vertical extent 143 of approximately 2 inches to 8 inches, or the back vertical extent 143 may comprise approximately 10% to 90% of a total vertical extent of the back side 114, or may comprise approximately the entire total vertical extent of the back side 114.

With continued reference to FIGS. 1-9 and FIGS. 10A-10G, the apparatus 100 further comprises a power source 130. The power source 130 may comprise a rechargeable battery capable of providing electrical power to the first set of heating channels 120, the second set of heating channels 122, and if present, the other sets of heating elements. Advantageously, the power source 130 may be recharged by USB connection, or other connection now known or later invented. The power source 130 is electrically connected with a controller 132 comprised in the apparatus 100, utilizing a power connector 135. The controller 132 is electrically connected, utilizing a heating connector 137, with, depending on the aspect of the present disclosure, the first set of heating channels 120, the second set of heating channels 122, and if present, the other sets of heating elements, and/or with the heating fibers 150 in aspects of the garment 110 having the heating fibers 150. The controller 132 allows the user 200 to turn the first set of heating channels 120, the second set of heating channels 122, and if present, the other sets of heating elements on and off. In some aspects of the apparatus 100, the apparatus 100 may comprise more than one power source 130, and in such aspects, it may be advantageous to have a first power source 130a power one or more of the sets of heating channels and/or one or more of the heating fibers 150, and to have a second power source 130b power a different one or more of the sets of heating channels and/or a different one or more of the heating fibers 150. One advantage of such an aspect of the apparatus 100 is redundancy, and another advantage is more compact wiring if the heating channels and/or heating fibers 150 are distributed in different areas of the garment 110. There may be any number of such power sources in the apparatus 100.

In some aspects of the present disclosure, the controller 132 may comprise a multiple-position switch 133. In other aspects of the present disclosure, the controller 132 may comprise one or more switches 134, typically two or more switches such that the present disclosure teaches a switch from the one or more switches 134 for each of the first set of heating channels 120, the second set of heating channels 122, and if present, the other sets of heating elements. In some aspects of the present disclosure, the controller 132 may further comprise an individual control 136 for each of the first set of heating channels 120, the second set of heating channels 122, and if present, the other sets of heating elements. This enables the user 200 to individually control each of the first set of heating channels 120, the second set of heating channels 122, and if present, the other sets of heating elements, to deliver heat and relief of period pain and discomfort to the areas of the body of the user 200 that are painful. Each individual control 136 may be a potentiometer, or other element to allow variable and user-controlled delivery of electrical power to the heating channel that the user 200 wishes to heat. Alternatively, each individual control 136 may offer multiple user-selectable levels of power to the first set of heating channels 120, the second set of heating channels 122, and if present, the other sets of heating elements.

In some aspects of the present disclosure, the power source 130 may be removably placed and stored in a pocket 138, wherein the apparatus 100 comprises the pocket 138. The pocket 138 may advantageously be incorporated into the garment 110, such that the garment 110 comprises the pocket 138. The power source 130 may be placed into the pocket 138 and then electrically connected to the controller 132, and the foregoing is reversible such that the power source 130 may be disconnected from the controller 132 and removed from the pocket 138, whereupon the garment 110 may be washed without damaging the power source 130. In some aspects, the power source 130 may be waterproof such that it could be washed with the garment 110. The pocket 138 may be closed with any pocket-closure component 139 that is suitable for this purpose, including but not limited to zippers, buttons, snaps, and hook-and-loop closures.

Certain aspects of the present invention were described above. From the foregoing it will be seen that this invention is one well adapted to attain all the ends and objects set forth above, together with other advantages, which are obvious in and inherent to the inventive apparatus disclosed herein. It will be understood that certain features and sub-combinations are of utility and may be employed without reference to other features and sub-combinations. It is expressly noted that the present invention is not limited to those aspects described above, but rather the intention is that additions and modifications to what was expressly described herein are also included within the scope of the invention. Moreover, it is to be understood that the features of the various aspects described herein are not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

Accordingly, what is claimed is:

1. An apparatus for heating relief of period pain, the apparatus comprising a garment, wherein the garment comprises:
a fabric;
at least a front side and a back side, together a set of one or more sides;
at least a first set of heating channels; and
a power source, the power source being electrically connected with a controller utilizing a power connector, and the controller being electrically connected utilizing a heating connector with the at least first set of heating channels.

2. The apparatus of claim 1, wherein the garment further comprises two or more sets of heating channels, wherein each of the two or more sets of heating channels are located in any of the set of one or more sides.

3. The apparatus of claim 2, wherein any of the two or more sets of heating channels are positioned near to a lower abdomen region of a user.

4. The apparatus of claim 3, wherein the two or more sets of heating channels comprise the first set of heating channels located in the front side, and a second set of heating channels located in the back side.

5. The apparatus of claim 2, wherein the two or more sets of heating channels are arranged in a pattern.

6. The apparatus of claim 2, wherein the two or more sets of heating channels comprise materials selected from a group including but not limited to carbon fiber materials and metallic thread.

7. The apparatus of claim 2, wherein the apparatus comprises more than one power source, being at least a first power source and a second power source, wherein the first power source powers the first set of heating channels, and wherein the second power source powers a second set of heating channels.

8. The apparatus of claim 1, wherein the fabric comprises heating fibers, and wherein the controller is electrically connected utilizing the heating connector with the heating fibers.

9. The apparatus of claim 8, wherein the apparatus comprises more than one power source, being at least a first power source and a second power source, wherein the first power source powers a first area of the garment made of the fabric, and wherein the second power source powers a second area of the garment made of the fabric.

10. The apparatus of claim 2, wherein the controller comprises a multiple-position switch and/or the controller comprises two or more switches wherein each of the switches controls one of the two or more sets of heating channels.

11. The apparatus of claim 1, wherein the garment further comprises a pocket, and wherein the power source can be reversibly placed and stored in the pocket, and removed from the pocket.

12. The apparatus of claim 4, wherein the first set of heating channels have a front horizontal extent of approximately 10% to 90% of a total horizontal extent of the front side, and the first set of heating channels have a front vertical extent of approximately 10% to 90% of a total vertical extent of the front side; and wherein the second set of heating channels have a back horizontal extent of approximately 10% to 90% of a total horizontal extent of the back side, and the second set of heating channels have a back vertical extent of approximately 10% to 90% of a total vertical extent of the back side.

13. An apparatus for heating relief of period pain, the apparatus comprising a garment, wherein the garment comprises:
a fabric, where at least a portion of the garment is made with the fabric, and wherein the fabric comprises heating fibers, which are woven or otherwise made or attached into some or all of the fabric;
at least a front side and a back side, together a set of one or more sides; and
a power source, the power source being electrically connected with a controller utilizing a power connector, and the controller being electrically connected utilizing a heating connector with the fabric.

14. The apparatus of claim 13, wherein the fabric comprised of the heating fibers comprises a first area located in the front side, and a second area located in the back side.

15. The apparatus of claim 14, wherein the heating fibers comprised in the first area are arranged in a first pattern, and wherein the heating fibers comprised in the second area are arranged in a second pattern.

16. The apparatus of claim 14, wherein the fabric comprised in the first area is woven with a first amount of the heating fibers per unit area of the fabric, and wherein the fabric comprised in the second area is woven with a second amount of the heating fibers per unit area of the fabric.

17. The apparatus of claim 14, wherein the fabric comprised in the first area is woven with at least a first type of heating fibers, and wherein the fabric comprised in the second area is woven with at least a second type of heating fibers.

18. The apparatus of claim 14, wherein the first area has a front horizontal extent of approximately 10% to 90% of a total horizontal extent of the front side, and the first area has a front vertical extent of approximately 10% to 90% of a total vertical extent of the front side; and wherein the second area has a back horizontal extent of approximately 10% to 90% of a total horizontal extent of the back side, and the second area has a back vertical extent of approximately 10% to 90% of a total vertical extent of the back side.

19. The apparatus of claim 14, wherein the controller comprises a multiple-position switch and/or the controller comprises two or more switches wherein each of the switches controls one of the areas of the fabric.

20. The apparatus of claim 13, wherein the heating fibers comprise materials selected from a group including but not limited to carbon fiber materials and metallic thread.
